(19) United States
(12) Patent Application Publication
(10) Pub. No.: US 2005/0065455 A1
(43) Pub. Date: Mar. 24, 2005

(54) Blood-collection device

(76) Inventor: Walter Sarstedt, Numbrecht (DE)

Correspondence Address: THE FIRM OF KARL F ROSS, 5676 RIVERDALE AVENUE, PO BOX 900, RIVERDALE (BRONX), NY, 10471-0900, US

(21) Appl. No.: 10/499,747

(22) PCT Filed: Dec. 16, 2002
(86) PCT No.: PCT/DE02/04587

(30) Foreign Application Priority Data
Dec. 21, 2001 (DE) 101 63 719.5

(51) Int. Cl. A61B 5/00

(52) U.S. Cl. 600/577

(57) ABSTRACT

The invention relates to a device for receiving bodily fluids, comprising a collection tube (1), whose front end is equipped with a domed section (7) with a pierceable stopper for an attachable guide sleeve (2), which on the side facing the domed section has a cannula (5) equipped with a rubber valve (3) and on the side facing away from the domed section has a connecting piece or the front section of a double cannula. The domed section (7) has a zone that can be deformed inwards and the exterior wall of the domed section in said zone and/or the interior wall of the guide sleeve (2) in a complementary zone have or has a projecting contour in relation to the remaining exterior zone of the domed section (7) and/or in relation to the remaining interior zone of the guide sleeve (2), said contour deforming the wall of the domed section (7) radially inwards, when the deformation-resistant guide sleeve (2) is attached. The domed section and the guide sleeve form in their combined usage position a non-positive fit, whose retaining force is greater than restoring force of the rubber valve (3).

The invention relates to a device for drawing body fluids, having a specimen tube having at an outer end a tip with a pierceable plug for a guide sleeve fittable on the tip and provided on its side turned toward the tip with a needle and with an elastomeric needle-shield tube and on the side turned away from the tip with a connection fitting or the opposite end of a double needle.

Such devices are for example used for drawing blood from a bottle or bag or to take a specimen from a connection vessel of for example urine. In every case there is the problem that the elastomeric needle-shield tube that surrounds the needle and that is collapsed like a bellows when the guide sleeve is fitted to or installed on the tip exerts a substantial sprint return force acting against the forces that retain the guide sleeve on the tip, with the result that the guide sleeve is pushed off the tip. In order to get around this problem, various measures are taken.

In the blood-drawing device described in German 3,049,503, the cap closing the outer end of the specimen tube has a cylindrical axially extending tip. The tip is closed at its outer end by a pierceable plug that is trapped between an inner centrally apertured wall of the tip and an outer-end rim. The tubular guide sleeve, that has on its outer end a holder for a double-ended and pointed needle whose outer end is intended for insertion into a vein while its inner end projects fo far into the guide sleeve that when the guide sleeve is fitted to the specimen tube it pokes through the plug, is axially shiftable and rotatable on the tip. The inner end of the needle projecting from the guide tube is contained in a bag-like tube (elastomeric needle-shield tube) of such length that the inner point of the needle does not initially reach to its closed end.

In order that the guide sleeve stays on the tip in spite of the spring pressure from the elastomeric needle-shield tube the tip is provided with a laterally projecting bump for holding the double-ended needle that fits in an angled slot in the guide sleeve. This holding bump projecting over the slot in the periphery forms a sort of bayonet latch that secures the guide sleeve to the double needle. Such a latch ensures a solid connection of the fitted-together parts of the blood-drawing device, but increase it s production cost. In addition the coupling and decoupling or latching of the guide sleeves requires that the holding bump first be aligned by turning of the specimen tube with the closing screw or plug cap to align with the slot, which requires some adept manipulation so that the parts can be properly aligned.

German 692 25 609 describes a protective housing for a needle screwed into a needle holder. Here the protective housing is rotatable on the holder to which end the protective housing has a ring forming an inwardly open groove in which a ridge on a tip of the holder fits.

It is an object of the invention to provide a device of the above-described type with a simple and reliable connection for the two interfitting parts that can be produced at low cost, is easy to use, and provides a solid enough retention to resist the spring force (return force) of the needle-shield tube tending to open it.

This object is achieved by a first embodiment with the features of claim 1, by a second embodiment with those of claims 2, and by another embodiment with those of claim 3.

All embodiments recognize the basic idea that for example standard retaining formations facility a grip of the guide sleeve and the tip of the plug or screw cap, but do not provide a solid seat, either too tight or too lose, between the two fitted-together parts of the device so that one does not get an acceptable connection hold when coupling to or disconnecting from a specimen tube. This problem is cured by the system of this invention when for example in a starting position before installation of the guide tube, the tip has in what will be its connection at least one retaining formation (such as a longitudinal rib, ridge, bump, or the like) or is of outwardly convex or barrel shape or the inner surface of the guide sleeve (see claim 2) is appropriately shaped, or both such systems are used, or there is a lesser wall thickness or a softer material so that when the hard and rigid guide sleeve is slipped into places the radially projecting retaining formation or the projecting shape exerts radially inwardly effecting elastic forces that are also applied to the guide sleeve such that the guide sleeve is retained against the return or spring forces on the tip.

In further embodiment according to the invention the retaining formations projecting outward from the surface of the tip are deformed elastically to the side in that the longitudinal ribs or for example closely juxtaposed bumps or short webs or similar formations are pushed about their longitudinal axes laterally and angularly fo the tip. Thus in spite of the relative small diameter of the interfitting parts (guide sleeve and tip) the desired self-locking hold is achieved, the parts fitting together more easily and with less resistance and similarly separating more easily than a standard tapered luer connection.

The gripping that is the result of the deformation of the tip or the shape of the tip near its retaining formations or the projecting outer shape and/or the guide sleeve as a result of the lateral deflection of the retaining formations from their normal positions or the deflected retaining formations with prestress is such that the return force exerted the needle-shield tube does not push off the guide sleeve. The temporary radial deformation caused by the diameter relationships either of the tip or the lateral deflection of the holding formations caused by the rigid guide sleeve can be set by appropriate selection of materials and dimensioning with respect to size and elasticity.

There is thus always an interaction between a rigid guide sleeve with the tip and a diameter difference between these two parts such that in the connection region there is a solid enough grip. This is ensured in that the tip is made elastically deformable, for example by using an appropriate plastic and/or dimensioning its wall thickness such that its wall deflects radially inward when the guide sleeve is fitted to it. The over sizing and deflection of the elastically yielding tip ensures that no matter what the inside diameter of the guide sleeve which has for example longitudinal ribs, a ridge, or a local raised part, is smaller than the outside diameter of the tip that is thus cylindrical and is deflected inward by the strength of the guide sleeve. On the other hand when the retaining formations deflect laterally, the tip and the guide sleeve are both rigid and nondeformable.

Further embodiments and particular features of the invention are seen in the claims and the following description of embodiments of a blood-drawing device according to the invention as shown in the drawing. Therein:

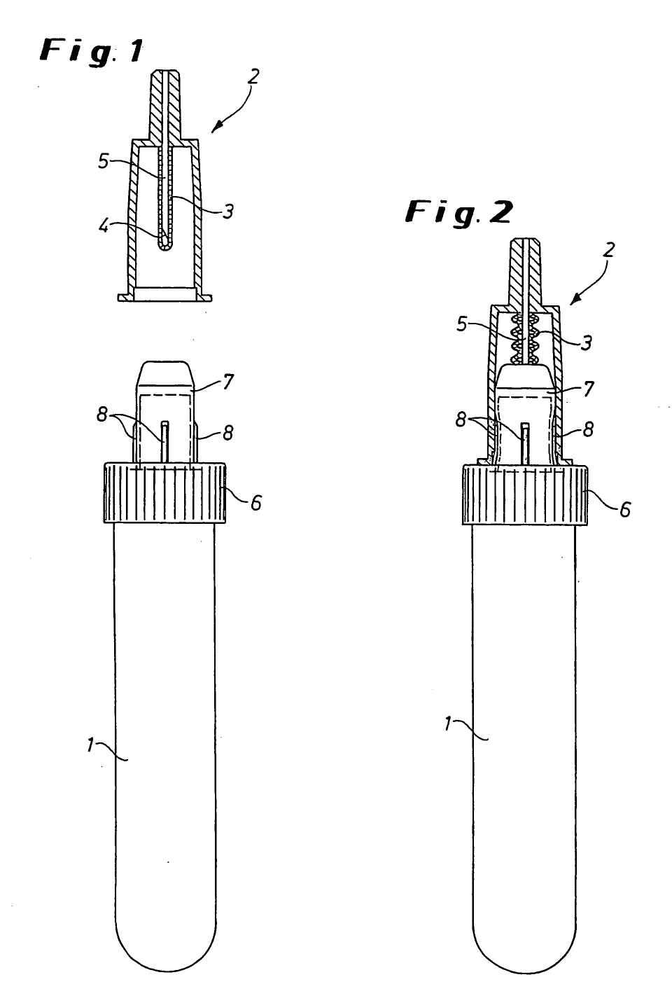

FIG. 1 is an exploded view of a specimen tube with a guide sleeve;

FIG. 2 is an overall view of the guide tube fitted over the tip of the specimen tube of FIG. 1;

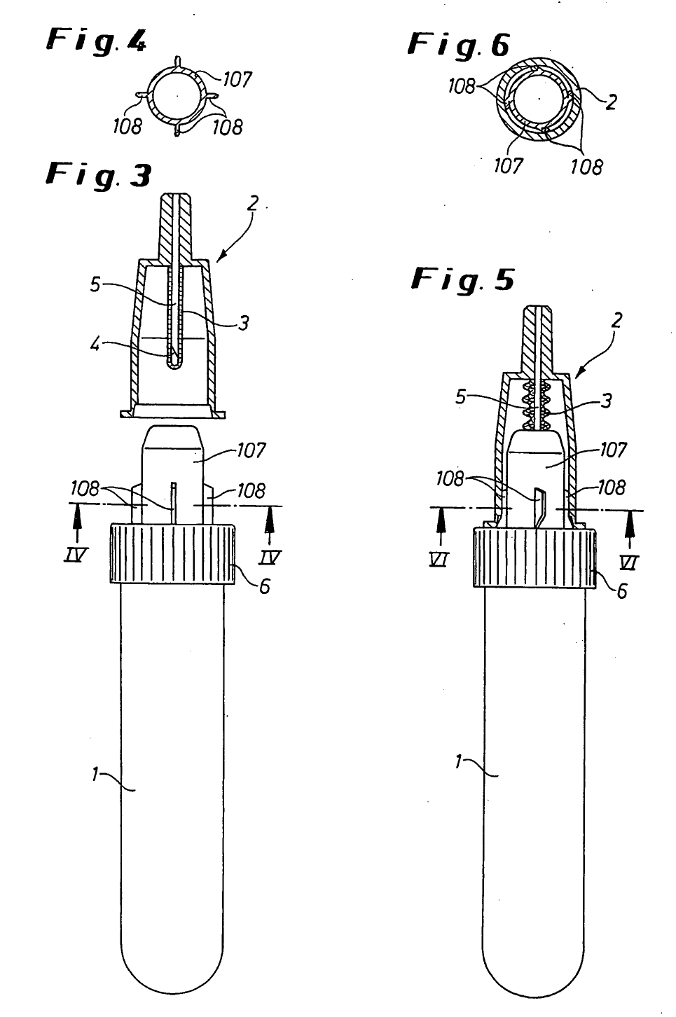

FIG. 3 is an exploded view of another embodiment of the specimen tube and guide sleeve;

FIG. 4 is a section taken along line IV-IV of FIG. 3;

FIG. 5 is an overall view of the guide tube fitted over the tip of the specimen tube of FIG. 3; and

FIG. 6 is a section taken along line VI-VI of FIG. 5.

A blood-drawing device according to FIGS. 1 and 2 comprises a specimen tube 1 with a tip 7 or 107 and a guide sleeve 2 provided in all embodiments for example with a luer fitting and holding a needle 5 having a point 4 and surrounded by an elastomeric shield tube 3. In other embodiments of the guide sleeve, its side turned away from the tip has a connector or the outer part of a double needle. The guide sleeve 2 is made of a rigid hard plastic. The specimen tube 1 is closed at its upper end by a cap 9 that has the cylindrical tip 7. It has in this embodiment a plurality of retaining formations 8 provided spaced about its periphery on its outer surface, here shaped as longitudinal ribs that start about in the middle of the tip and go to its inner end. Alternatively the holding formations can be for example rows of closely spaced bumps or the like or similar formations on the outside surface of the tip and/or the inside surface of the guide sleeve, for example either convex or concave. In the connection region provided with this shape or these retaining formations 8 the diameter is oversize, that is the outside diameter of the tip 7 is larger than the inside diameter of the guide sleeve 2. Such an oversized diameter between the guide sleeve 2 and tip 7 is also provided when in a manner not shown here the inside diameter of the guide sleeve is partially smaller than the outside diameter of the tip.

To draw blood the guide sleeve 2 and the specimen tube 1 are pushed together, that is the rigid guide sleeve 2 is slid onto the tip 7 of the cap 6. At first the end of the elastomeric shield tube 3 engages the plug set in the tip 6. On further pushing-together, the shield tube 3 collapses like a bellows (see FIG. 2) and is pierced by the inner needle point 4 which then pokes through the plug so that the point 4 is exposed inside the specimen tube. Once the rigid guide sleeve 2 is in its end position of FIG. 2 in contact with the retaining formations 8 (here longitudinal ribs), the guide sleeve 2 will bear radially inward via the retaining formations 8 on the elastically deformable wall of the tip 2 and deform it radially inward into a waisted shape as shown in FIG. 2. The guide sleeve 2 is thus held with prestress solidly on the tip 7 of the cap 6.

This holding effect is also present when, according to the embodiment of FIGS. 3 to 6 where reference numerals from the described embodiment are applied to corresponding structure, the tip 107 retains its original shape when fitted with a rigid guide sleeve 2 and instead the retaining formations or longitudinal ribs 108 are constructed such that they deflect when the guide sleeve 2 is fitted in place, for example pushing angularly to the side of the tip against its surface and away from the inner surface of the guide sleeve, that is pivoting about their longitudinal axes 9 as shown in FIGS. 5 an 6. The tip 107 is here rigid like the guide sleeve 2 while the retaining formations 1208 are elastically resilient.

The embodiments of FIGS. 1 and 2 an 3 to 6 have in common that without any change in the inside diameter of the guide sleeve as it is fitted over the cap as a result of the material it is made of and its dimensions with the retaining formations relative to the guide-sleeve inside diameter and the over sizing of the tip when unstressed, there is either a radial inward deformation of the tip in the connection region of the retaining formations deflect laterally. The necessary holding force can also be obtained when the inside diameter of the rigid guide sleeve is slightly smaller than the outside diameter of the tip so that its walls also must deflect inward or the dom is provided with a convex shape (e.g. rounded or barrel-shaped).

1. A device for drawing body fluids, the device comprising a specimen tube (1) having at an outer end a tip (7) with a pierceable plug for a guide sleeve (2) fittable on the tip (7) and provided on its side turned toward the tip (7) with a needle (5) and with an elastomeric needle-shield tube (3) and on the side turned away from the tip with a connection fitting or the outer end of a double needle,
characterized in that
the tip (7) has an inwardly elastically deformable region and the outer surface of the tip in this region and the complementary inner surface of a rigid guide sleeve (2) have relative to the rest of the outer surface of the tip (7) and the rest of the inner surface of the guide sleeve (2) such a shape that on mounting of the rigid guide sleeve (2) the wall of the tip (7) deforms radially inwardly, a connection being formed between the fitted-together tip and guide sleeve that is strong enough than return force of the shield tube (3).

2. A device for drawing body fluids, the device comprising a specimen tube (1) having at an outer end a tip (7) with a pierceable plug for a guide sleeve (2) fittable on the tip (7) and provided on its side turned toward the tip (7) with a needle (5) and with an elastomeric needle-shield tube (3) and on the side turned away from the tip with a connection fitting or the outer end of a double needle,
characterized in that
the tip (7) has an inwardly elastically deformable region and the inner surface of a rigid guide sleeve (2) has relative to the rest of the inner surface of the guide sleeve (2) such a shape that on mounting of the rigid guide sleeve (2) the wall of the tip (7) deforms radially inwardly, a connection being formed between the fitted-together tip and guide sleeve that is strong enough than return force of the shield tube (3).

3. A device for drawing body fluids, the device comprising a specimen tube (1) having at an outer end a tip (107) with a pierceable plug, the tip (107) having on its outer surface at least one radially projecting retaining formation (108) for a guide sleeve (2) fittable on the tip (107) and provided on its side turned toward the tip (107) with a needle (5) and with an elastomeric needle-shield tube (3) and on the side turned away from the tip with a connection fitting or the outer end of a double needle,
characterized in that
the tip (107) including at least one of its retaining formations (108) is oversized in a connection region relative to an inside diameter of a rigid guide sleeve (2) and the retaining formations (108) is constructed such that on fitting of the rigid guide sleeve (2) it deflects laterally and in a fitted-together use position of the guide sleeve (2) and tip (107) the holding force is greater than the return force of the shield tube (3).
